(19) United States
(12) Patent Application Publication
(10) Pub. No.: US 2020/0188171 A1
(43) Pub. Date: Jun. 18, 2020

(54) System for Treatment of Meibomian Gland Dysfucntion

(71) Applicant: Danelli Ocular Creations, LLC, Granger, IN (US)

(72) Inventors: Bryan Minelli, Savannah, GA (US); Marco Minelli, Mishawaka, IN (US); Kevin Danahey, Granger, IN (US)

(21) Appl. No.: 16/715,142

(22) Filed: Dec. 16, 2019

Related U.S. Application Data
(60) Provisional application No. 62/781,224, filed on Dec. 18, 2018.

(51) Int. Cl. A61F 9/007; A61F 7/00

(52) U.S. Cl. CPC A61F 9/00772; A61F 2007/0095; A61F 2007/0004; A61F 7/007

(57) ABSTRACT

The present system is directed to the treatment of Meibomian gland disorder, specifically, clearing the blockage of the Meibomian glands on the eyelids. The system disclosed herein enables heating the inner eyelid, compressing the outer eyelid, and exfoliating the eyelid border at the same time to promote the clear flow of meibum onto the eye.

### CROSS-REFERENCE TO RELATED APPLICATIONS

This application incorporates by reference and claims the benefit of priority to U.S. Provisional Application 62/781,224 filed on Dec. 18, 2018.

### BACKGROUND OF THE INVENTION

The present invention relates to a system, method, and apparatus for the treatment of Dry Eye Disease. More particular, the present invention relates to treating Meibomian Gland Dysfunction and Blepharitis, two of the most commonly associated causative factors in Dry Eye Disease.

Dry Eye Disease (DED) is now considered a major global health concern. The prevalence of DED is increasing and estimates show that approximately 30 million people in the United States alone are affected with this condition. This disease is expected to increase and will create a significant societal and economic burden on our healthcare system in the United States and globally. Factors such as increasing age, the use of digital devices, increases in chronic metabolic diseases such as diabetes, hypertension, autoimmune diseases, cancer, and the medications used to treat them all play a causative role. The use of contact lenses as well as environmental factors and hormonal changes also play a significant role. DED has a significant impact on quality of life and is a main contributing factor for loss of productivity in the workplace. DED causes difficulties with everyday activities such as computer use, reading, driving, and watching television. Pharmaceutical treatments for this disease are available however these medications are very expensive and create an enormous economic burden for insurance companies and patients while creating a major barrier to access for the millions of patients who suffer from this disease. These medications also do not address the most common etiology of DED, Meibomian Gland Dysfunction (MGD) and Blepharitis.

Recent clinical studies have shown that the most common cause of DED is from evaporative disease and Meibomian Gland Disease (MGD) is the primary cause of this condition. It has been estimated that up to 86% of patients with DED have MGD. Since most patients with DED have MGD, it seems reasonable to question why billions of dollars are spent on treatments that do not address the root cause of this disease. It is therefore imperative to create innovative solutions for this disease while decreasing the socioeconomic burden of DED and improving the quality of lives of the patients who are afflicted with condition.

Meibomian glands are sebaceous glands that are located on the lid margin of both the upper and lower lids. There are approximately 25 to 40 meibomian glands in the upper lid and 25-30 in the lower lid. Miebomian glands are responsible for the production of the outermost layer of the tear film. This layer consists of fatty acids and lipids known as meibum. This oily layer provides an important role in the function of the visual system. This protective layer of lipids prevents the evaporation of the tear film from the ocular surface and is necessary for optimal visual acuity. Failure to maintain an optimal lipid layer results in rapid evaporation of the tear film from the ocular surface resulting in blurred and fluctuating vision as well as symptoms such as burning, tearing, itching, photophobia, and infection.

MGD is a multifactorial disease that can cause the meibum to become thickened over time which ultimately causes the glands to become blocked, plugged, and obstructed. If left untreated, MGD will cause permanent atrophy, gland dropout, and irreversible loss of the meibomian glands.

Blepharitis is another condition strongly associated with MGD. Blepharitis is a condition that causes inflammation of the eyelids and eyelid margins. The three main causes of blepharitis are staphylococcal, seborrheic, and demodex. Studies show that up to 50% of the adult population has some form of blepharitis and recent studies show that 100% of patients over 70 have demodex blepharitis. The inflammation from these forms of blepharitis lead to a cascade of events that ultimately create the presence of a biofilm on the lid margin. This biofilm and keratinization of the eyelid margin further lead to obstruction of the meibomian glands contributing to gland atrophy and permanent loss of the glands. Symptoms of blepharitis, MGD, and DED all have overlapping symptoms such as blurred vision, tearing, itching, a sandy gritty feeling, and light sensitivity. These symptoms are so closely tied together that many experts and leaders in the industry are calling blepharitis, MGD, and DED one term known as Dry Eye Blepharitis Syndrome (DEBS).

Now that research has uncovered major root causes of DED, it is incumbent upon clinicians, researchers, and innovators to address MGD and Blepharitis and create treatment strategies that address both of these conditions concurrently. In order for treatments to be effective, innovative technologies should be created that focus on the three contributing factors of DED: 1. Obstruction of the meibomian glands. 2. Removal of the biofilm on the eyelids and eyelid margins. 3. Addressing inflammation.

Many traditional and emerging treatments are available for the treatment and management of MGD and Blepharitis but as will be outlined below have significant disadvantages to the unique and novel approach we are proposing for our solution. Many of the current treatment strategies are cost prohibitive and creates a barrier to access. Others are not effective, time consuming, and create compliance issues.

One of the most common and traditional approaches for MGD and Blepharitis are the use of warm compresses, eyelid massage, and eyelid scrubs. Warm compresses have significant limitations as the heat has to travel through tissue, muscle, and fat in order to create enough heat to effectively melt the meibum to a sufficient level. In addition, this is time consuming for the patient and compliance is a major issue. Eyelid scrubs with baby shampoo and now commercially available preparations also play a role in current treatment; however, there are limitations with these home hygienic treatments. Compliance, improper technique, inability of some patients to properly touch the lid margin can lead to drop out. Although these do play an important role in maintaining the health of the lid margin and removal of the biofilm, they do not address obstruction and inflammation of the meibomian glands.

Topical antibiotics and steroid combinations have also been widely used in the treatment of blepharitis and MGD. These treatments however are not without significant side effects. Long term use of these pharmaceuticals can cause antibiotic resistance as well as glaucoma and the formation of cataracts. This causes further economic impact as surgery may be necessary to remove the cataracts caused by the steroid drops or ointments. Oral antibiotics such as doxycycline have been widely used and do have a role in addressing the inflammation associated with MGD, however there can be significant side effects such as photosensitivity, GI distress, and can also affect the health of the intestinal microbiome.

Another treatment for MGD is in office expression of the meibomian glands. This entails squeezing the affected eyelid in order to remove the meibum from the obstructed glands. This can be done with the use of a cotton tip applicator, an eyelid paddle, or finger. These techniques are variable and dependent on the physician and can be very painful for the patient. In addition, there is no way to measure the amount of force the physician is applying to the eyelid. This method of expression does not address inflammation or removal of the biofilm.

Intense Pulse Light (IPL) is an emerging treatment for MGD and has shown to be effective. This method of treatment uses a specific wavelength of light around the eyelid margins through a mechanism not fully understood, but appears to address the obstruction of the meibomian gland. Limitations to this procedure are the significant costs to the patients and providers. In addition, careful evaluation of the skin type has to be of primary concern as patients with more pigmentation can develop significant burns and create unnecessary liabilities to the physicians. In addition, four to six treatments are necessary in order to achieve effective results. These treatments are typically done three to four weeks apart so most patients do not notice improvement until several months have passed.

Another in office treatment for blepharitis is Blephex, which includes a small handheld electromechanical device performs a microblepharoexfoliation of the eyelid margins using disposable microsponges. The Blephex treatment can be useful for removal of the biofilm and keratinization of the lid margin but does not express or remove the meibum from within the channel of the meibomian gland. It is often used in conjunction with another in office procedure using vectored thermal expression there by having the patient undergo two separate procedures and the physician having to invest in two medical devices in order to accomplish a comprehensive treatment that embodies addressing obstruction, inflammation, and removal of the biofilm.

Yet another in office procedure available to treat MGD is the LipiFlow system developed by Tear Science and now owned by Johnson and Johnson™. This system involves placing a heating element between the inner eyelid and eyeball and an automated external controller maintains the heat to a level sufficient to melt, soften, and loosen the meibomian glands. In addition, pneumatic inflatable bladders are placed on the exterior of the lid margin while applying a predetermined pattern of time pulsed compression on the external eyelid margins during an approximately twelve minute period. Although this system has been shown to be an effective treatment for MGD, it has several disadvantages. First the cost of the procedure is very expensive for patients and once again provides a barrier to access. In addition, the cost to the physician for the LipiFlow equipment and disposables can be very cost prohibitive and as a result, the majority of physicians are unable to offer this treatment to their patients. Other limitations are as follows. The treatment is a one size fits all approach. The physician can't control the amount of pressure applied to the meibomian glands and the glands may not reach the required pressure to effectively express the glands in a meaningful way. It does not allow the physician to customize the treatment based on the level of disease. The machine is not ideal for transporting to satellite offices due to its expense and size limitations. Although it is effective at addressing obstruction of the meibomian glands, it does not address removal of the biofilm. This biofilm is usually removed with another medical device prior to treatment which requires the physician to pay for another medical device. In addition, the patient has to have two separate procedures. Lastly, it does not address the inflammatory component of the disease.

The most recent medical device used in the treatment of MGD is Alcon's ILux system. This method uses an external LED light source to heat the meibomian glands and positions a back plate between the eyelid and eyeball while positioning a compressive element on only a portion of the external lid margin and applying a compressive force between the eyelid and back plate while viewing through an aperture. This application has many disadvantages. First, the heat is from an external source proximate to the outside of the lid margin. This heat must travel through muscle, tissue, and fat to reach the meibomian glands located on the inner eyelid. It also takes 40 seconds to heat each zone and when the temperature reaches the appropriate heat, the light goes off allowing the glands a chance to cool while the device is compressing only a small portion of the lid margin. In order to effectively treat each eye, a physician has to move the device to three zones on the upper and lower eyelid of each eye while having to wait the full 40 seconds for each zone to heat to the appropriate temperature. This requires six separate treatments for each eye and can be extremely time consuming for both patient and provider. It requires a significant amount of dexterity as the handheld device must be turned upside down in order to treat the upper lids. In addition, there is no way for the physician to gauge how much pressure (psi) one is exerting on the lid margins which can make the treatment inconsistent among physicians. This treatment is also expensive due to the high cost of the disposables and once again provides a difficult barrier to entry into the DED market. Lastly, this device does not address the removal of the biofilm and once again requires the physician to invest in other technologies and burden the patient with more procedures and costs.

Therefore, a need therefore exists for a method and treatment apparatus for MGD that effectively embodies treating all aspects of this condition. The needed device would address all three components of treating this disease including meibomian gland obstruction, removal of the biofilm, and addressing inflammation in one comprehensive solution while addressing the present challenges and limitations such as those discussed above.

### BRIEF SUMMARY OF THE INVENTION

The present disclosure provides systems and methods for treating Meibomian gland dysfunction. Various examples of the systems and methods are provided herein.

The present system is directed to the treatment of Meibomian gland disorder, specifically, clearing the blockage of the Meibomian glands on the eyelids. The system disclosed herein enables heating the inner eyelid, compressing the outer eyelid, and exfoliating the eyelid border at the same time to promote the clear flow of meibum onto the eye.

Specifically, the system includes a soft, mechanical vibration applied to both the upper and lower lid margins simultaneously. The application of the vibration aids in the loosening and removal of the obstructed meibum in the meibomian glands to help restore sufficient lipid flow to the tear layer. The vibrations also assist in the exfoliation of the lid margins from obstructive material such as debris, blepharitis, and capped meibomian glands. A compressive force can be applied from the outside of the patient's eye lids. The application of force expresses the obstructed meibomian glands during and after the exfoliation is concluded by mechanically evacuating the meibum from the orifices of each meibomian gland. A shielding device can be placed between the patient's eyeball and eyelids which protects the eye from a heating element. The heating element applies heat to the inner surface of the eyelid to a temperature level sufficient to cause the meibum to soften, loosen, and melt throughout the channels of each meibomian gland. The application of heat aids in restoring adequate meibum secretion.

In an example, a method of treating meibomian gland dysfunction includes a vibrating source placed between the patient's upper and lower lid margins to exfoliate the obstructive material such as debris, blepharitis and capping of the meibomian gland orifices. The method can include maintaining the vibration for a period of time necessary to exfoliate lid margins, and maintaining the vibration for a period of time necessary to loosen and break down obstructed meibum. The method can include the expression of the obstruction from the meibomian glands by applying a controlled force to the outside of a patient's eyelid. The expression of the meibomian glands may be performed before, during, and after the vibration is applied and maintained for a period of time. The method can include repeatedly applying and maintaining vibration to the patient's eyelids to exfoliate and maintain the obstruction loosened, repeatedly applying a controlled, compressive force to the patient's eyelids for a period of time necessary to express obstruction, and maintaining vibration for a period of time. The method can include compression of the glands and exfoliation of lid margins. The meibomian glands can be heated from an inner heating source.

An advantage of the present device includes providing heat to the inner eyelid provides semi-direct and/or indirect contact to the Meibomian glands while compression of the outer eyelids promotes the flowing of the Meibum.

An advantage of the present device includes an exfoliation brush that provides cleansing and removal of bacterial biofilm, keratinized epithelium, dead skin, and any blockage within and/or on the eyelids.

An advantage of the present device is providing heating, compression, and exfoliation to the eyelids at the same time in one device.

A further advantage of the present system is providing a device with removable and replaceable eyepiece elements to provide a device that can be reused while maintaining hygiene and preventing the spread of any eye infections. In an example, the system is reusable up to 6-12 months or more.

Another advantage of the present device includes providing user control of the pressure, temperature, exfoliation, and treatment duration, including instant start-stop treatment. As a result, the present system provides a patient customized treatment of eye conditions.

The present device provides a hand held assembly to maneuver the device to access the pertinent glands in a more efficient way.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a side view of an example of the device disclosed herein.

FIG. 2 is a back view of an example of the device.

FIG. 3 is a top view of an example of the device.

FIG. 4 is a cross section view of an example of the removable inner eyepiece.

FIG. 5 is a front view of an example of the removable outer eyepiece.

FIG. 6 is a cross section of an example of the exfoliating brush element.

FIG. 7 is a cross section of an example of the device in combination with a patient's eye.

### DETAILED DESCRIPTION OF THE INVENTION

The present systems and methods for treating Meibomian gland dysfunction using a device for stimulating the Meibomian glands of the eyelids. The device can provide heat, vibration, and pressure to a user's eyelid to release the blockage and restore the adequate function of the Meibomian glands.

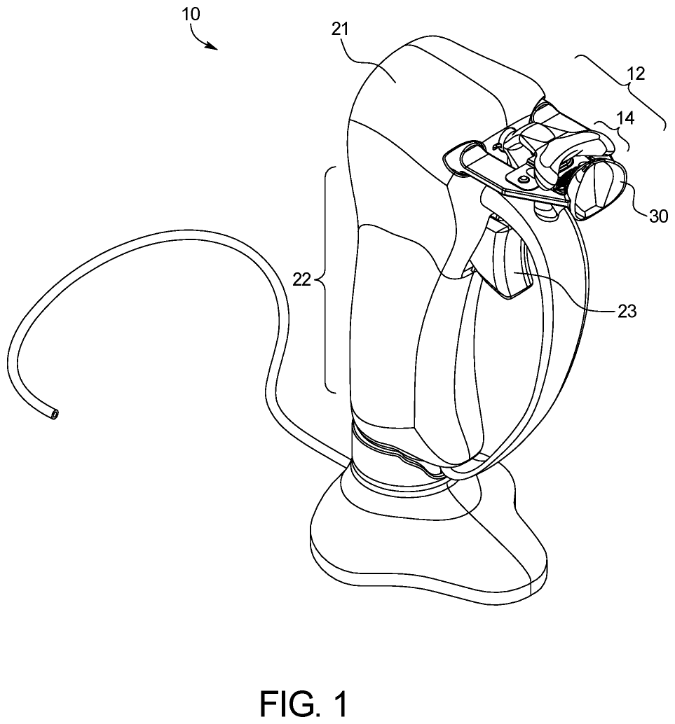

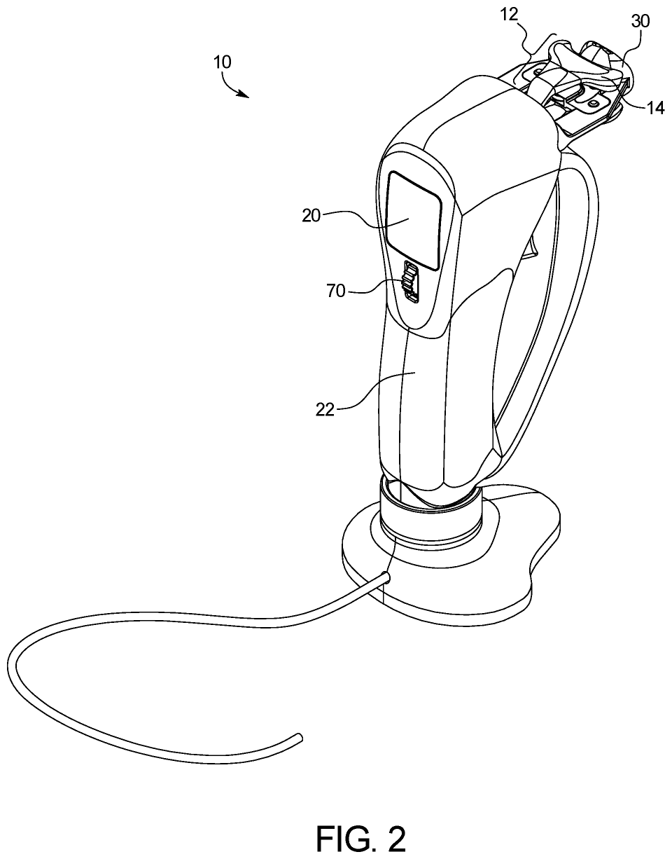

As shown in FIG. 1, the system 10 can include an outer eyepiece 12, wherein the outer eyepiece can include a concave surface 14 that can be used to apply pressure to an outer eyelid surface. As shown in FIG. 5, the outer eyepiece 12 can include at least one pressure sensor 16 for determining the amount of pressure applied to the eyelid via the outer concave surface 14 of the outer eyepiece 12. However, the position of the pressure sensor can be in any suitable position (i.e., the position is not limited to the arrangement in FIG. 5). The pressure sensor 16 can be in communication with a controller, wherein the controller can display the pressure sensor measurement on a user interface 20, as shown in FIG. 2.

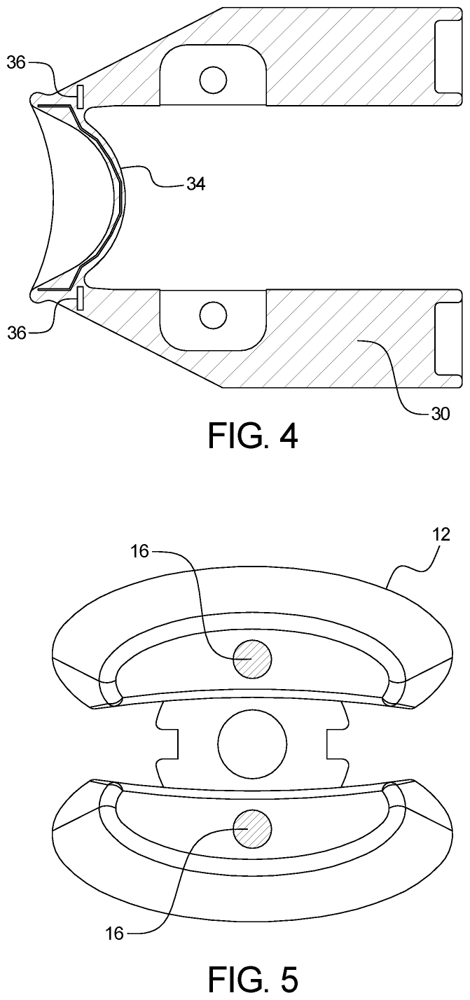

The system 10 can include a removable concave inner eyepiece 30 that counteracts the pressure from the outer eyepiece. The inner eyepiece 30 connects to the outer eyepiece 12, wherein the inner eyepiece 30 can extend from the outer eyepiece 12. As shown in FIG. 4, a user's eye can be pressed against the concave surface of the inner eyepiece 30. The inner eyepiece 30 can include at least one temperature control mechanism that can heat the inner eyepiece. The temperature control mechanism can include a heating element 34 to control the temperature of the inner surface of the inner eyepiece 30. The system 10 can also include a temperature sensor 36 that can be in communication with the inner eyepiece 30 to determine and communicate the temperature measurement of the inner eyepiece 30 to the controller. The controller can display the temperature measurement on the user interface 20.

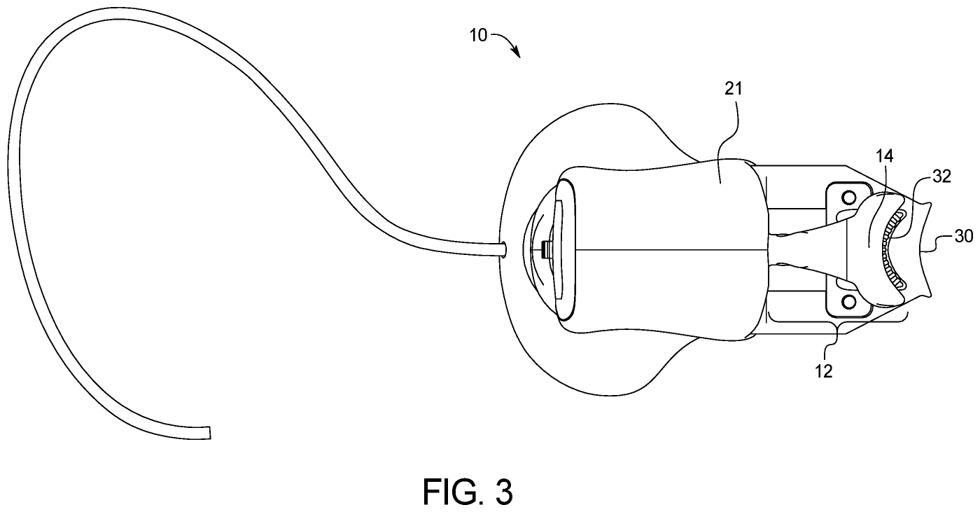

As shown in FIG. 3, the inner eyepiece 30 and/or the outer eyepiece 12 can include an exfoliating element 32. For example, the exfoliating element 32 can include a microbrush that can be vibrated via a motor 40 in communication with the microbrush. The inner eyepiece 30 can be composed of a flexible polymeric material, soft elastomeric material, a silicone material, or combinations thereof, among others.

As shown in FIG. 2, the device 10 can include a user interface 20 for displaying the sensor results. For example, the user interface 20 can display the temperature measured by the temperature sensor, the pressure measured by the pressure sensor, the time of application to the eyelid, the status of the vibrating microbrush, among other data. The system 10 can include a button, switch, or touch screen on the user interface for selection to activate the vibrating microbrush via the vibrating motor. For example, FIG. 2 illustrates an example with a selector wheel 70. The user interface 20 can be located on or within a handle 22 of the device. However, the user interface 20 can be in a separate device, such as a smart device, external computer, etc. In an example, the system 10 can provide a software application in communication with the controller, wherein the application can provide a user interface for a user to control the treatment variables of the system 10 (e.g., duration, temperature, pressure, exfoliation, etc.) The acquired and generated data from the system 10 and/or controller can be communicated to the application to display the results to the user. The system 10 can provide a patient-specific (e.g., customized treatment) treatment plan. For example, the system 10 can collect personal information from the patient, and based on the personal data, the system 10 can generate a treatment protocol that can be communicated to the device for treatment.

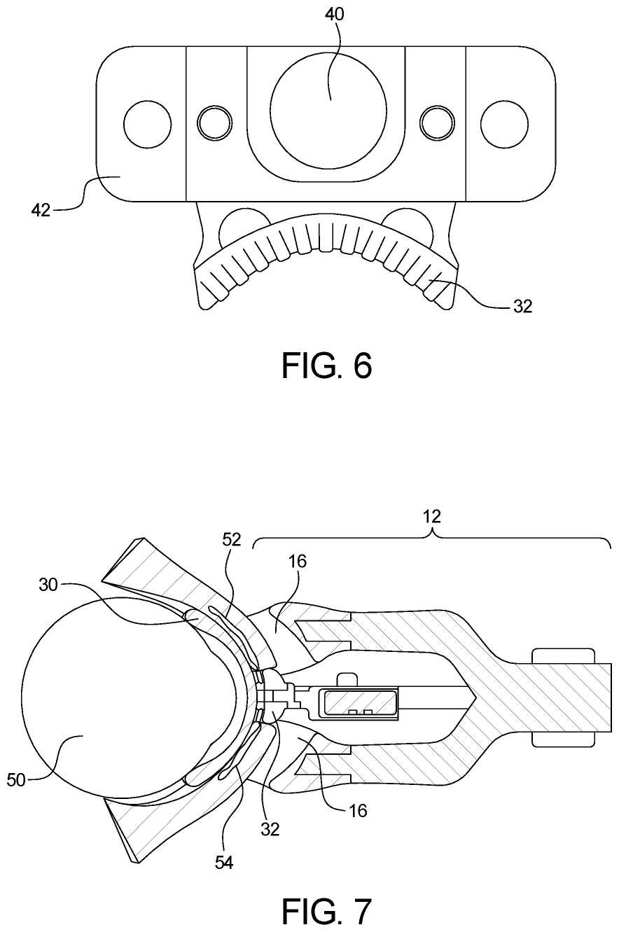

FIG. 6 is a cross section of a motor enclosure 42 that can be positioned within the outer eyepiece 12. The vibrating motor 40 can be in communication with the exfoliating element 32, such that the motor 40 controls the vibration of the exfoliating brush element 32. The exfoliating element 32 (e.g., microbrush) can be comprised of a cellulose sponge material. A supporting material for the microbrush can be used to connect to the vibrating motor 40. In addition, a vibration dampening element can be positioned between the motor 40 and the inner eyepiece 30.

The system 10 can include a controller in communication with the sensors and user interface 20. The controller can be in communication with a memory for storing the device application data. The system 10 can include a power source, for example, a rechargeable battery to power the device. In an example, the system 10 can include a charging base that can be used to recharge the power source of the device. The system 10 can include an indicator of power charge level, for example, on the user interface and/or a separate red/green light to indicate the charge level of the power source. In addition, the system 10 can indicate which eye should be treated.

The system 10 can include a handle 22, wherein the outer eyepiece can attach to a head 21 of the handle 22. The handle 22 can be used to position the device on the user's eye, wherein a trigger 23 on the system 10 can be used to control the application of pressure to an eye and/or eyelid. For example, as shown in FIG. 7, when the system 10 is positioned on a user's eye 50 the inner eyepiece 30 is in contact with the user's eye, specifically the upper Meibomian gland 52 and the lower Mebomian gland 54, without direct contact to a user's cornea, and the outer eyepiece 12 slides to contact the user's outer eyelid.

The present disclosure also includes a method of treating Meibomian gland dysfunction including providing a system 10 including two eyepieces. For example, the system 10 can include a removable outer eyepiece 12 that provides a compressive force to the outer surface of the user's eyelid, thus providing expression, and an inner eyepiece 30 for applying heat, pressure, and exfoliation to a user's eyelid. In an example, the inner eyepiece 30 can include an exfoliating element 32 for exfoliation of the eyelid. The method removes any blockages and forces the Meibomian glands to excrete, thereby, restoring the gland's proper function.

The method includes applying heat via the inner eyepiece to the eyelid to a predetermined temperature. The temperature can be controlled via the controller through the user interface 20. The controller can report and monitor the time passed during the application of the heat. For example, after a predetermined time of heat application, the system 10 can notify the user that the user can remove the device from the user's eyelid. In an example, the temperature range can include 90-120° F., for example, 107-109° F. (41.6-42.7° C.). The increased temperature can be applied between 1-20 minutes, for example, 5-15 minutes.

The method can include applying the exfoliating element, for example, a microbrush. The method can include applying the exfolating element to the pores on the eyelid border to loosen, agitate, and break up any blockages in the gland. The brush is applied to the closed eyelid. In an example, the exfoliating element can be positioned on the border of the user's eyelid when the inner eyepiece is positioned on the outer surface of a user's eye ball and the outer eyepiece is positioned on the outer surface of the user's eyelid. The vibrating brush can be activated independently from the heating application. The vibration duration and intensity can depend on the severity of the patient's level of keratinization and biofilm formation. After and during vibration, the blockage can be deposited on the brush that can be discarded and replaced before the subsequent treatment. The meibum that is expressed from the application of the device can be deposited onto the ocular surface and subsequently drained into the puncta and absorbed by a sponge. In an example, the treatment can include application of an ocular anti-microbial liquid solution to alleviate irritation of the eyelid.

As mentioned above, aspects of the systems and methods described herein are controlled by one or more controllers. The one or more controllers may be adapted to run a variety of application programs, access and store data, including accessing and storing data in the associated databases, and enable one or more interactions as described herein. Typically, the controller is implemented by one or more programmable data processing devices. The hardware elements, operating systems, and programming languages of such devices are conventional in nature, and it is presumed that those skilled in the art are adequately familiar therewith.

For example, the one or more controllers may be a PC based implementation of a central control processing system utilizing a central processing unit (CPU), memory and an interconnect bus. The CPU may contain a single microprocessor, or it may contain a plurality of microprocessors for configuring the CPU as a multi-processor system. The memory may include a main memory, such as a dynamic random access memory (DRAM) and cache, as well as a read only memory, such as a PROM, EPROM, FLASH-EPROM, or the like. The system may also include any form of volatile or non-volatile memory. In operation, the memory stores at least portions of instructions for execution by the CPU and data for processing in accord with the executed instructions.

The one or more controllers may also include one or more input/output interfaces for communications with one or more processing systems. Although not shown, one or more such interfaces may enable communications via a network, e.g., to enable sending and receiving instructions electronically. The communication links may be wired or wireless.

The one or more controllers may further include appropriate input/output ports for interconnection with one or more output mechanisms (e.g., monitors, printers, touchscreens, motion-sensing input devices, etc.) and one or more input mechanisms (e.g., keyboards, mice, voice, touchscreens, bioelectric devices, magnetic readers, RFID readers, barcode readers, motion-sensing input devices, etc.) serving as one or more user interfaces for the controller. For example, the one or more controllers may include a graphics subsystem to drive the output mechanism. The links of the peripherals to the system may be wired connections or use wireless communications.

Although summarized above as a PC-type implementation, those skilled in the art will recognize that the one or more controllers also encompasses systems such as host computers, servers, workstations, network terminals, and the like. Further one or more controllers may be embodied in a device, such as a mobile electronic device, like a smartphone or tablet computer. In fact, the use of the term controller is intended to represent a broad category of components that are well known in the art.

Hence aspects of the systems and methods provided herein encompass hardware and software for controlling the relevant functions. Software may take the form of code or executable instructions for causing a controller or other programmable equipment to perform the relevant steps, where the code or instructions are carried by or otherwise embodied in a medium readable by the controller or other machine. Instructions or code for implementing such operations may be in the form of computer instruction in any form (e.g., source code, object code, interpreted code, etc.) stored in or carried by any tangible readable medium.

As used herein, terms such as computer or machine “readable medium” refer to any medium that participates in providing instructions to a processor for execution. Such a medium may take many forms. Non-volatile storage media include, for example, optical or magnetic disks, such as any of the storage devices in any computer(s) shown in the drawings. Volatile storage media include dynamic memory, such as the memory of such a computer platform. Common forms of computer-readable media therefore include for example: a floppy disk, a flexible disk, hard disk, magnetic tape, any other magnetic medium, a CD-ROM, DVD, any other optical medium, punch cards paper tape, any other physical medium with patterns of holes, a RAM, a PROM and EPROM, a FLASH-EPROM, any other memory chip or cartridge, or any other medium from which a controller can read programming code and/or data. Many of these forms of computer readable media may be involved in carrying one or more sequences of one or more instructions to a processor for execution.

It should be noted that various changes and modifications to the embodiments described herein will be apparent to those skilled in the art. Such changes and modifications may be made without departing from the spirit and scope of the present invention and without diminishing its attendant advantages. For example, various embodiments of the systems and methods may be provided based on various combinations of the features and functions from the subject matter provided herein.

We claim:

1. A system for stimulating the Meibomian glands of an eyelid of a user, the system comprising:
a body including a head portion and a handle portion extending from the head portion, wherein the body includes at least one pressure sensor;
an outer eyepiece including an outer concave surface, wherein the outer eyepiece extends from the head portion of the body; and
an inner eyepiece extending from the outer eyepiece, wherein the inner eyepiece includes an inner concave surface for application to an eye surface of the user, wherein the inner eyepiece includes a temperature sensor on the inner concave surface,
wherein at least a portion of the outer concave surface moves relative to the inner eyepiece to apply pressure to an outer surface of the eyelid of the user,
wherein the inner eyepiece includes an exfoliating element in communication with a vibrating motor for vibrating the exfoliating element, wherein the exfoliating element is positioned along an eyelid border of the user.

2. The system of claim 1, further comprising a trigger mechanism attached to the handle, wherein the trigger mechanism controls a pressure applied to an eyelid via the outer eyepiece.

3. The system of claim 1, further comprising a heating element in communication with the temperature sensor.

4. The system of claim 3, wherein the inner eyepiece includes the heating element.

5. The system of claim 1, wherein the exfoliating element includes a microbrush.

6. The system of claim 1, wherein the exfoliating element includes a microbrush, wherein the microbrush includes a cellulose sponge material, a silicone material, or combinations thereof.

7. The system of claim 1, wherein the inner eyepiece includes a flexible polymeric material, a soft elastomeric material, a silicone material, or combinations thereof.

8. The system of claim 1, further comprising a user interface on a portion of the body, wherein the user interface displays at least one of a temperature measured by the temperature sensor, a pressure measured by the pressure sensor, and a time of application of the device to the eyelid.

9. The system of claim 1, further comprising a vibrating motor in the body, wherein the vibrating motor is in communication with the exfoliating element.

10. The system of claim 1 further comprising
a controller;
a memory coupled to the controller, wherein the memory is configured to store program instructions executable by the controller;
wherein in response to executing the program instructions, the controller is configured to
receive user input via a user interface in communication with the controller, wherein the user input corresponds to a temperature, a pressure, and a duration time for application of the device to the eyelid of the user, heat the heating element to the received temperature of the user input, apply the received pressure of the user input to the outer eyepiece, and vibrate the exfoliating element for the received duration time.

11. A method of treating Meibomian gland dysfunction, the method comprising:
providing a device including
a body including a head portion and a handle portion extending from the head portion, wherein the body includes at least one pressure sensor;
an outer eyepiece including an outer concave surface, wherein the outer eyepiece extends from the head portion of the body, and
an inner eyepiece extending from the outer eyepiece, wherein the inner eyepiece includes an inner concave surface for application to an eye surface of the user, wherein the inner eyepiece includes a temperature sensor on the inner concave surface,
wherein at least a portion of the outer concave surface moves relative to the inner eyepiece to apply pressure to an outer surface of the eyelid of the user,
wherein the inner eyepiece includes an exfoliating element in communication with a vibrating motor for vibrating the exfoliating element, wherein the exfoliating element is positioned along an eyelid border of the user;
positioning the inner eyepiece on an outer surface of an eye of the user;
positioning the outer eyepiece on an outer surface of the eyelid of the user;
applying a compressive force via the outer eyepiece to the eyelid of the user; and
exfoliating the border of the eyelid of the user via the exfoliating element to express blockages in a Meibomian gland in the eyelid of the user.

12. The method of claim 11, further including applying heat to the eye of the user via a heating element in the inner eyepiece.

13. The method of claim 11, wherein the heat is provided at a temperature in a range of 40 to 45° C.

14. The method of claim 11, wherein the heat is applied in a range of 1 and 20 minutes.

15. The method of claim 12 further comprising
receiving user information via a user interface;
generating a treatment plan including a prescribed pressure, a prescribed temperature, and a prescribed duration of application of the device to the eyelid of the user;
heating the heating element to the prescribed temperature;
applying the prescribed pressure to the eyelid of the user via the outer eyepiece; and
vibrating the exfoliating element for the prescribed duration time.

16. The method of claim 15 further comprising displaying the treatment plan on the user interface.
